(19) United States
(12) Patent Application Publication
(10) Pub. No.: US 2026/0317371 A1
(43) Pub. Date: Oct. 15, 2026

(54) Protein Recovery Method

(71) Applicant: Renix Inc., London (CA)

(72) Inventors: David Andrew Prince, London (CA); Long Sang, London (CA)

(21) Appl. No.: 19/156,934

(22) PCT Filed: Feb. 7, 2024
(86) PCT No.: PCT/CA2024/050146

§ 371 Date: Aug. 15, 2025

Related U.S. Application Data
(60) Provisional application No. 63/485,100, filed on Feb. 15, 2023.

(51) Int. Cl. C07K 1/18

(52) U.S. Cl. CPC C07K 1/18

(57) ABSTRACT

A steady-state process for the recovery of soluble protein from an aqueous protein-containing feed fluid is provided. The process comprises the steps of: i) passing protein capture particles in a flow countercurrent to the feed fluid in an adsorber at steady state pH to yield protein-loaded particles; ii) transferring the protein-loaded particles from the adsorber into a desorber; iii) passing said protein-loaded particles in a flow countercurrent to a protein recovery eluent in the desorber to desorb said protein from said particles at steady state pH to provide protein-free particles; iv) collecting the desorbed protein from the desorber; v) transporting the protein-free particles to a regenerator, and regenerating the particles; and vi) feeding the regenerated particles into the adsorber for use to repeat steps i)-v) in a continuous manner. An apparatus useful to conduct the steady-state process is also provided.

### FIELD OF THE INVENTION

This invention generally relates to processes for the recovery of protein from by-product, co-product or waste streams arising from plant or biomass material processing, and in particular, relates to a process for direct recovery of proteins from such streams as a high purity protein ingredient.

### BACKGROUND

The global demand for protein is increasing at a dramatic rate, driven by population growth in developing parts of the world. At the same time, climate change and resource stresses in agriculture are putting pressure on traditional methods of protein production, while competition for protein not only for food, but for animal feed and aquaculture is also growing. In addition, consumer behaviour with respect to protein sources is changing due to concerns related to health, climate change, the environment, and animal welfare. As a result, in many countries there is rapidly growing interest in alternative proteins such as plant-based protein.

Plant or biomass material is composed of a range of components, including for example starches and sugars, fibre, fats, oils, proteins, and ash (an inclusive term used to represent the total small amounts of many inorganic compounds such as metals and minerals, among others). Global processors and manufacturers in food & beverage, feed, nutraceutical, flavour & preservative, biofuel & biochemical, cosmetics, cleaning, pharmaceuticals, and other industries use plant or biomass material feedstocks to produce thousands of products. In the production of these products, the plant or biomass material is generally separated into one or more of its components. For example, in the food industry, corn is processed to generate corn oil, high fructose corn syrup, sugars, sweeteners, biofuels and biochemicals. In the beverage industry, barley, wheat, rye, oats, sugarcane, corn, fruit and other materials are used in fermentation processes to produce beer, wine, spirits, cider, kombucha, kefir, tepache and other products. In another example, sugar sources are used in fermentations to produce organic acids for a variety of purposes such as cleaning products or preservatives or to replace fossil fuel derived chemicals. Plants or biomass may also be specifically cultivated for the purpose of producing specific niche products, such as those used in nutraceuticals, flavours & preservatives.

In the process of making these products, there are often by-product, co-product or waste streams generated at various processing steps that may be depleted of one component, but still contain other valuable products such as proteins. While the cultivation of high-protein crops such as soybeans or peas is a commonly used approach to providing plant-based protein, increasing protein production would require dedication of agricultural resources which are already under pressure. A process for protein recovery from existing sources would contribute to increasing protein supply without adding demand for additional agricultural space or related resources. In producing protein ingredients from such plant or biomass material, the challenge is to develop a process to separate proteins from other components in such as way as to provide a pure protein that is suitably functional for use in intended products, while also being technically robust, economical, and sustainable.

Processes used to isolate proteins from plant or biomass-based sources often include separation of starches and sugars, fibre and/or oil via a variety of unit operations and protein purification using processes such as hydrolyzing steps, coagulation and/or precipitation through the application of heat and/or pH adjustment, as well as solid-liquid separation such as filtration, centrifugation, decantation or similar operations. Many of these processes have a significant impact on protein solubility.

Other separation processes include the use of sorption-based operations such as for example adsorbents, zeolites, chromatography and/or ion exchange to separate and purify the proteins following the removal of other components. For example, Segall (WO02/089597) describes the production of canola 2S protein from residual oil seed meal using a process based on ion-exchange chromatography. Modinger (WO2016/001683A1) describes a process for producing proteins arising from distillation processes, based on the use of a high-silica content adsorption matrix to bind protein found in by-product streams from distillation processes. Markedal (WO2012116703A1) describes a process for manufacturing products including protein from a plant material using heat, pH adjustment, centrifugation, and heated acidic suspension followed by precipitation processes and the use of Sepharose cationic ion exchange chromatography resins to separate proteins from non-protein components.

The use of adsorbent, zeolite, chromatography or ion exchange-based processes for producing specialized high-value proteins such as those used in pharmaceutical or nutraceutical applications has been implemented at production scale. However, using these processes for producing proteins for food, feed or other use has seen limited implementation industrially due to several inherent challenges in scale-up of these processes. Some challenges include: i) high capital and operating costs associated with existing batch-style adsorbent, zeolite, chromatography and ion exchange platforms driven by resin capacities which require frequent regeneration and the associated high volumes of chemicals and water; ii) high cost of downstream purification, concentration and/or drying steps due to low product stream purity and/or concentration; iii) large physical and environmental footprints associated with existing batch-style adsorbent, zeolite, chromatography and ion exchange platforms; iv) difficulty controlling pH-driven agglomeration or precipitation of protein in resin or membrane systems which can cause irreversible plugging of beds or membranes, damage equipment or cause system shut downs; and v) costs associated with controlling microbial growth in large volumes of dilute biomass material.

Fluidized or expanded beds assist with some of these challenges and have found application in ion exchange processes. For example, Lihme (WO2008086811A1) describes a method of providing a purified protein product from a raw material used in the production of biofuel, based on creating a material suspension and subjecting that suspension to an expanded bed adsorption process to obtain a purified protein product. Haas et al. (PCT/CA2012/000417) describe an apparatus for an ion exchange platform that provides a steady-state operating environment, however, the application of this platform for soluble protein recovery is not described.

Thus, it would be desirable to develop a process to recover soluble protein from bi-product, co-product or waste streams such as a plant or biomass material-based stream at production scale.

### SUMMARY OF THE INVENTION

A process for the recovery of soluble protein from an aqueous feed fluid has now been developed which can be effectively applied at production scale. The process utilizes a steady-state system adapted for protein recovery.

Thus, in one aspect of the invention, a steady-state process for the recovery of soluble protein from an aqueous protein-containing feed fluid is provided comprising the steps of:
i) passing protein capture particles in a flow countercurrent to the feed fluid in an adsorber at steady state pH to yield protein-loaded particles;
ii) transferring the protein-loaded particles from the adsorber into a desorber;
iii) passing said protein-loaded particles in a flow countercurrent to a protein recovery eluent in the desorber to desorb said protein from said particles at steady state pH to provide protein-free particles;
iv) collecting the desorbed protein from the desorber;
v) transporting the protein-free particles to a regenerator, and regenerating the particles; and
vi) feeding the regenerated particles into the adsorber for use to repeat steps i)-v) in a continuous manner.

In another aspect, an apparatus for use to recover soluble protein at high purity from an aqueous protein-containing feed fluid, said apparatus comprising:
a first column for housing a first fluidized bed, said first column comprising a first particle-feeding inlet to feed protein capture particles into an upper region of said first column and a first fluid-feeding inlet to feed a first protein-containing fluid into a lower region of said first column to form a fluidized bed in the first column, said first column further comprising a first fluid outlet in the upper region at a height above the first particle-feeding means, said lower region being remote from said upper region such that said particles and said feed fluid flow counter-currently, wherein the upper region comprises a first end of the first column and the lower region comprises a second end of the first column;
a second column for housing a second fluidized bed, said second column comprising a second particle-feeding inlet to feed particles into an upper end of said second column and a second fluid-feeding inlet to feed a protein recovery fluid into a lower end of said second column to form a second fluidized bed in the second column, said second column further comprising a second fluid outlet at the upper end of the second column at a height above the second particle-feeding means, said upper and lower ends being remote such that said particles and said fluid flow counter-currently through said second column;
a first conduit which connects the first and second fluidized beds such that the second end of said first column is connected to the upper end of said second column, said first connecting conduit being adapted to permit passage of particles and prevent or minimize passage of said first protein-containing fluid into said second column;
a second conduit which connects the lower end of said second column to a transport section, said second connecting conduit being adapted to permit passage of particles and prevent or minimize passage of said protein recovery fluid into the transport section;
a regenerator to which the transport section connects and which is connected to the first particle-feeding inlet of the upper region of the first column via a third conduit, said transport section adapted for hydraulic transport of particles upwardly into the regenerator, which is for housing a third fluidized bed, said regenerator comprising a third particle-feeding inlet to feed particles into an upper end of said regenerator from the transport section and a third fluid-feeding inlet to feed a regenerant fluid into a lower end of said regenerator to form a third fluidized bed in the regenerator, said regenerator further comprising a third fluid outlet for removal of mineral-containing seffluent at the upper end of the regenerator at a height above the third particle-feeding inlet, said upper and lower ends being remote such that said particles and said fluid flow counter-currently through said regenerator; and
the third conduit which connects the lower end of said regenerator to the upper region of said first column, said third connecting conduit being adapted to permit passage of particles and prevent or minimize passage of said regenerant fluid into said first column.

These and other aspects of the invention are described in the detailed description and by reference to the following Figures.

### BRIEF DESCRIPTION OF THE FIGURES

FIG. 1 is a block diagram providing an overview of a protein purification scheme from aqueous protein-containing feed fluid which utilizes a method of protein recovery in accordance with an embodiment of the present invention;

FIG. 2 illustrates A) system sections and B) inlets and outlets of an apparatus within the system for use to conduct protein recovery according to the flowchart shown in C) in accordance with an embodiment of the invention;

FIG. 3 illustrates A) a table showing pKa and pI for 20 amino acids wherein pKa₁=α-carboxyl group, pKₐ₂=α-ammonium ion, and pKₐ₃=side chain group, and B) a table showing protein stability at different pH ranges;

FIG. 4 graphically illustrates protein recovery using a process in accordance with an embodiment of the invention using an eluent having varying salt concentrations;

FIG. 5 graphically illustrates the concentration and purity of protein recovered in accordance with an embodiment; and

FIG. 6 graphically illustrates the regeneration efficacy of an embodiment of the invention.

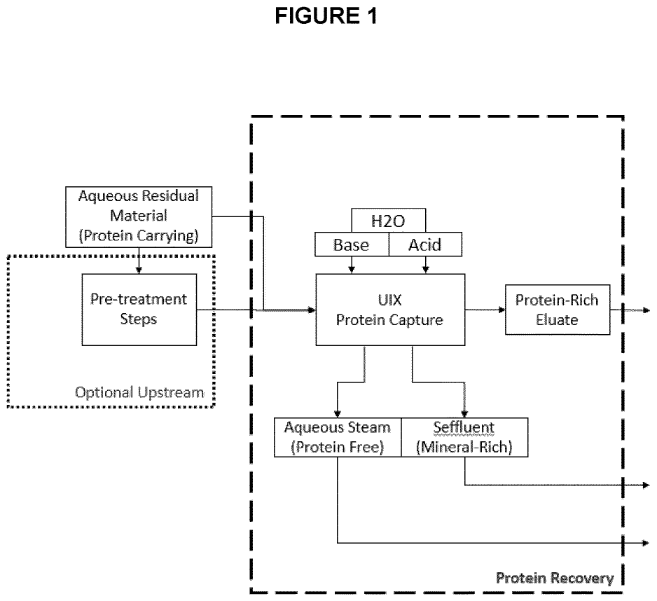

### DETAILED DESCRIPTION

A steady-state process for the recovery of soluble protein from a protein-containing feed fluid is provided. The process comprises the steps of: i) passing protein capture particles in a flow countercurrent to the feed fluid in an adsorber at steady state pH to yield protein-loaded particles; ii) transferring the protein-loaded particles from the adsorber into a desorber; iii) passing said protein-loaded particles in a flow countercurrent to a protein recovery eluent in the desorber to desorb said protein from said particles at steady-state pH to provide protein-free particles; iv) collecting the desorbed protein from the desorber; v) transporting the protein-free particles to a regenerator, and regenerating the particles; and vi) feeding the regenerated particles into the adsorber for use to repeat steps i)-v) in a continuous manner.

The term “steady state” as it is used herein with respect to the process of protein recovery refers to maintenance of the environment during the process, and during each stage of the process, in a state which essentially does not change over time, i.e. variables within the process are unchanging over time. Thus, steady state pH refers to maintenance of the pH throughout the process, including maintenance of pH changes during flow of feed fluid, eluent and regenerant fluids throughout the process. The term “essentially” is used herein to refer to the substantial maintenance of a given state, i.e. no significant change.

The protein-containing feed fluid for use in the present process may be any protein-containing fluid in aqueous solution having a pH of below 5 or above 7.5. In embodiments, the feed fluid may be derived from an aqueous protein-containing residual material that results from fermentation or processing of grains such as corn, wheat, maize, barley, rice and oats, or processing of other plant or agricultural biomass such as soybean, peas, potatoes, lentils, rapeseed, lupin, grass, common bean and cottonseed, to name a few sources of residual protein-containing material. The residual material may also arise from the separation of biomass into constituent components such as fibre, carbohydrates, oils or fats for the purpose of producing a product, using known processing technologies and/or processes such as for example extraction, precipitation, chromatography or membrane filtration. Depending on the original source of plant or biomass material, the processes employed upstream, and the nature of the fermentation or separation processes, the protein content of the residual material will vary, for example, from about 10-60%, and may be mixed with ash, carbohydrates, fiber, fats, oils, lipids, organic acids and/or other constituents.

The residual material from which the feed fluid is derived may optionally be pre-treated to reduce suspended solids content using technology conventionally used for this purpose including, for example, filtration, centrifugation or other methods. The residual material may additionally or alternatively be pre-treated to reduce dissolved mineral content using technologies well-established for this purpose such as, for example, adsorption, reverse osmosis (RO), distillation, precipitation or membrane filtration.

In a first step of the process, the protein-containing aqueous feed fluid is fed directly into a steady-state uninterrupted adsorber. The adsorber comprises a downward countercurrent flow of particles selected to capture the protein in the feed fluid as the feed fluid flows upwardly against the downward flow of the particles in the adsorber. The selected particles are chosen based on the target proteins and the nature and/or source of the protein-containing feed fluid, and thus, will vary based on the combination of these parameters, i.e. the plant or biomass material source and the employed upstream processing of the protein-containing feed fluid.

Suitable protein capture particles include ion exchange particles, adsorbent particles, chromatography particles and zeolite particles by which to separate proteins from a feed fluid to provide a high purity protein eluate. Ion exchange particles are typically based on a solid support such as a crosslinked polystyrene, polymethacrylate or polyacrylic matrix with functional ion exchange sites, either anionic (basic) or cationic (acidic), being attached to the support. As one of skill in the art will appreciate, the selection of the particle is based on the actual proteins and the presence of impurities in the feed fluid, the characteristics of the feed fluid, and the overall goals of the process.

In one embodiment, ion exchange is used to capture protein from the feed fluid. Ion exchange is a process for separating proteins in solution based on net charge. In this regard, the isoelectric point (pI) of the protein plays a role in determining the appropriate particles for use to capture the protein. The pI of a protein is the pH at which the protein has no net electrical charge or is electrically neutral in the statistical mean. The net charge of a protein will depend on the solution the protein is in. Thus, proteins are positively charged when in a solution having a pH below their pI and will require a cationic particle for protein capture. Examples of strong cationic exchange functionalized particles include sulfopropyl, sulfoethyl and sulfomethyl groups, and can include any ligand that contains a sulfonic acid group. Weak cationic ion exchange functionalized particles include carboxymethyl groups or any other ligands containing a carboxylate. Likewise, proteins are negatively charged at a pH above their pI and will require an anionic particle for protein capture. Examples of strong anionic ion exchange functionalized particles include trimethyl aminomethyl, trimethyl aminoethyl, diethyl-(2-hydroxypropyl) aminoethyl, trimethylamino-hydroxypropyl groups, and any other ligand containing a quaternary amine. Weak anionic ion exchange functionalized particles include diethyl amino or dimethyl amino groups, and any other ligand containing a tertiary amine, to capture negatively charged particles.

In one embodiment, the particles for use to capture protein in accordance with the invention are silica-free. Silica-free particles have been determined to be appropriate for use to isolate proteins, and do not pose limitations with respect to permitting protein capture in feed fluids.

As the feed fluid flows upwardly, protein is captured or bound to the selected downwardly flowing particles to yield protein-loaded particles, and ions released by the particles during this exchange enter the aqueous protein-free fluid. The released ions may change the pH of the aqueous fluid, for example by lowering the pH if the released ion is a hydrogen ion or by raising the pH if the released ion is a hydroxide ion. However, in the present process, the change in pH in the aqueous fluid is maintained constant within the adsorber as protein capture occurs. Thus, while the pH changes as the feed fluid flows upwardly against the downward flow of the particles in the adsorber, the pH profile within the adsorber is constant, thereby maintaining a steady-state pH profile, which is not achieved in a fixed bed purification system. This steady-state environment within the adsorber prevents or minimizes proteins from becoming uncharged at the wrong point in the profile which would render them unable to be captured by the particles. It also prevents or minimizes protein agglomeration and precipitation which can be highly detrimental to the system. The nature of the present process, i.e. the constant flow of the particles counter-currently to the upwardly flowing feed fluid, provides the steady-state operating environment that maintains constant chemistry of the process steps under steady-state conditions, thus allowing the adsorption step to occur in a stable pH profile.

The protein-free feed fluid, referred to as raffinate, is removed from the adsorber and may be additionally processed, discarded, or collected as a source of recycled water for other processes, optionally with additional processing such as pH adjustment prior to use.

As the protein-loaded particles exit the adsorber, they may be rinsed or washed with water or other solutions, and passed through a first dynamic seal. For the purposes of the present system, the term “dynamic seal” is used herein to refer to an arrangement to prevent the passage of one liquid into a second liquid through the control of pressures and mixing at the liquid/liquid interface. The dynamic seal is, thus, used to permit passage of protein-loaded particles while preventing the passage of protein-containing feed liquid from the adsorber into the desorber, as well as to adjust and control the solids circulation rate and system productivity.

The protein-loaded particles are then fed into the desorber in a downward flow countercurrent to an eluting protein-recovery fluid to result in desorption of the protein from the particles at steady-state pH to provide isolated protein at high purity for collection, and to provide protein-free particles.

The protein-recovery fluid is an eluent which is suitable to desorb or release the protein from the loaded particles. The protein-recovery fluid is selected based on the nature of the captured protein and the particles selected for protein capture, and will be sufficient to displace or desorb the protein from the particles. The protein-recovery fluid may be an acid (such as hydrochloric acid, sulfuric acid, phosphoric acid, nitric acid), a base (such as sodium hydroxide, potassium hydroxide, ammonium hydroxide), or a salt such as sodium chloride or potassium chloride. In the desorber, the dissolved ions from the selected protein-recovery fluid, generally hydrogen ions, hydroxide ions, or salt ions, displace the captured protein from the particle. The released protein is carried by the spent fluid stream (referred to as the eluate) out of the desorber and is collected in a holding tank for future processing, or fed directly for downstream processing which may include dewatering and/or drying to generate a final dried protein product. The isolated protein is of high purity, e.g. at least about 75% pure, preferably greater than 80%, 85% or 90% pure, such as 95% pure or greater.

Any dissolved ions resulting from the release of protein from the particles into the protein-recovery fluid may change the pH of the protein-rich eluate, lowering its pH if the released ion is a hydrogen ion, or raising its pH if the released ion is a hydroxide ion. However, as in the adsorber, the present process maintains the change in pH of the protein-containing eluate constant due to the operating environment of the process, thereby maintaining the steady-state pH profile within the desorber to prevent or minimize undesirable protein agglomeration and/or precipitation that would otherwise occur in the absence of the steady-state pH profile. Thus, in view of the operating environment of the present process, i.e. the constant flow of protein-loaded particles counter-currently to the upwardly flowing protein-recovery eluent, the chemistry of the desorption step is maintained under steady-state conditions, thus allowing protein desorption to occur in a stable pH profile.

The protein-free particles exit the desorber in steady-state. As the particles move out of the desorber, they may be rinsed or washed with water or other solution, and pass through a second dynamic seal which functions to prevent exit of protein-containing eluate from the desorber with the particle stream. The particles are transported to a regenerator section of the system.

The protein-free particles are fed into the regenerator and are flowed in a counter-current manner to a regenerant fluid. The regenerant functions to chemically condition the protein-free particles to return them to their original chemical form, i.e. a form sufficient for protein capture, in order for them to function effectively on re-entry into the adsorber. Thus, the regenerant may be an acid, base or salt, and will be based on the nature of the feed fluid and the adsorber particles. In the regenerator, ions bound on the particles (for example, minerals such as calcium, magnesium, potassium, sodium, chloride, and sulfate, and other entities such as organics), are released from the particles using the selected regenerant, generally acid, base or salts. Ions released from the particles are carried with the upwardly flowing spent regenerant (referred to as the seffluent) for removal from the regenerator. The mineral-rich seffluent may be transferred to a holding tank or fed directly for processing, e.g. acid or caustic recovery, or downstream processing, polishing and drying for the production of additional co-products.

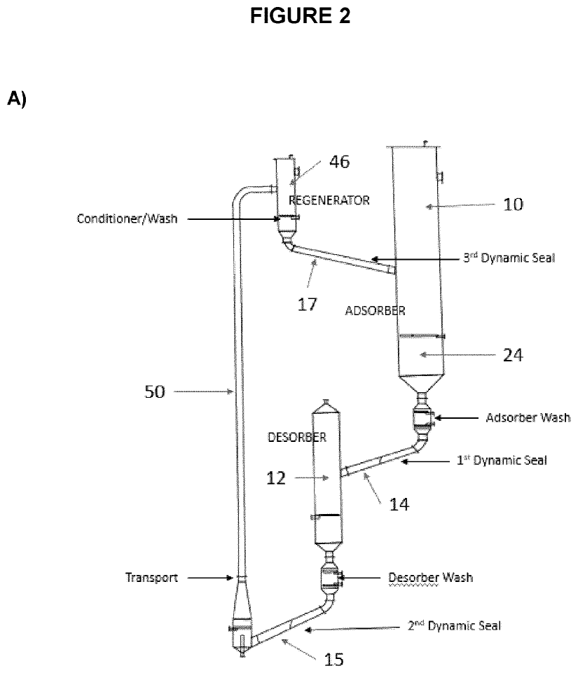

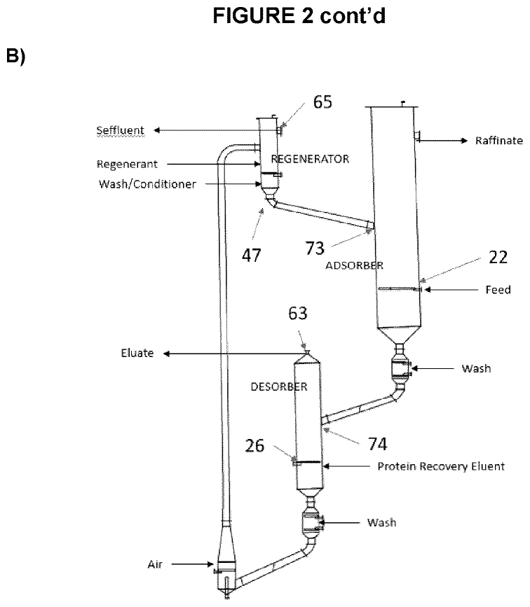

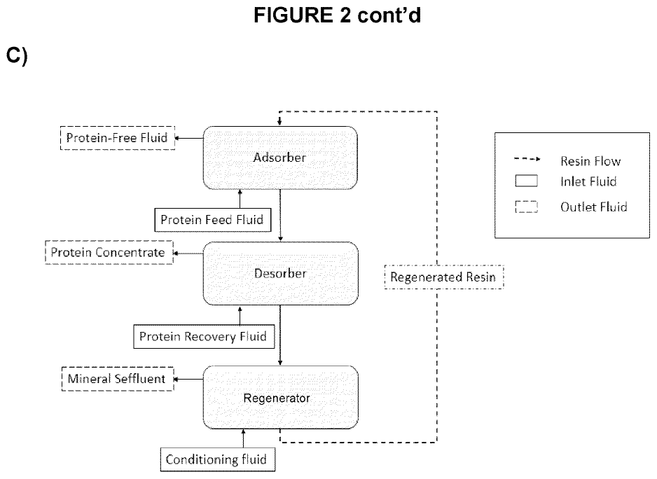

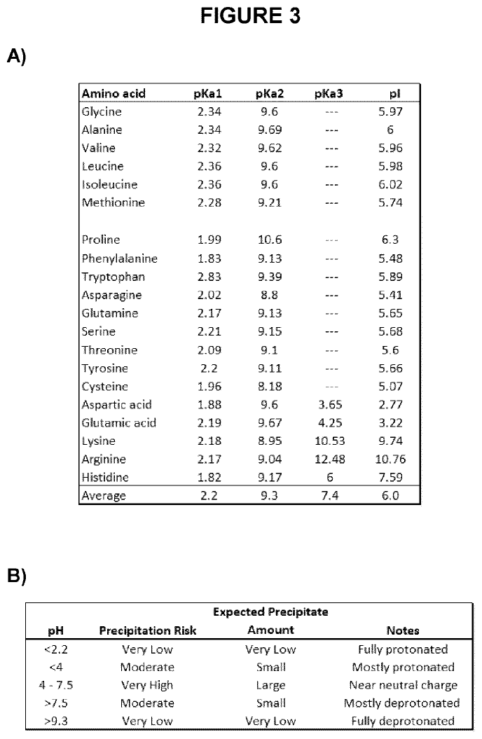

Once appropriately regenerated, the particles are flowed from the regenerator to the adsorber in steady state. As the particles move out of the regenerator, they may be rinsed or washed with water or other solution during transit so that they are prepared for use in the adsorber. The particles then move through a third dynamic seal which prevents passage of regenerant from entry into the adsorber with the regenerated particles. The regenerated particles then begin the cycle again, in steady state fashion, flowing downwardly counter-currently to incoming protein-containing feed fluid to adsorb protein therefrom, followed by protein desorption and collection, and particle regeneration, as illustrated in FIG. 2C).

The process is conducted under chemical and hydrodynamic conditions sufficient to achieve the effective capture of protein from a protein-containing feed fluid, and desorption of protein and other ions, as required. The adsorber section of the system is sized with a particle bed of sufficient volume to provide capacity and contact between the particles and protein-containing feed fluid to exchange all or substantially all of the protein from the feed fluid onto the particles based on the protein capacity of the selected resin and the protein type and its concentration within the protein-containing feed fluid, as would be appreciated by one of skill in the art. The desorber section is similarly sized with a particle bed of sufficient volume to provide capacity and contact between the particles and a protein-recovery fluid to exchange all or substantially all of the protein from the particles into the protein-rich eluate stream based on the selected protein recovery fluid, and the protein type and concentration. The regenerator section is sized with a particle bed of sufficient volume to provide capacity and contact between the particles and regenerant to exchange all or substantially all of the ions to be removed from the particles into the mineral-rich seffluent stream, as required, and to generate particles suitable for protein adsorption in the adsorber. Further, the rate at which the particles circulate through the system is selected to match the rate at which the feed fluid is intended to be processed and may be based on the nature of the feed fluid (e.g. protein concentration) as well as the nature of the protein capture particles and protein-recovery fluid.

The combination of these chemical and hydrodynamic conditions and the particle circulation rate controls the overall system chemistry in steady-state keeping operating conditions steady, thus controlling pH to manage precipitation and optimize productivity

Thus, the present method recovers protein directly from protein-containing feed fluid generated during plant or biomass-material processing and concentrates the protein at high purity for the purpose of creating beneficial protein products. The method is such that it provides a steady-state operating environment that controls the chemistry of the ion exchange steps without relying on low concentrations, thereby allowing each step to function at stable pH. This steady-state operating environment enables ion exchange chemistry to be used for protein separation by ensuring the proteins remain in charged state and further by limiting undesirable protein agglomeration and/or precipitation which generally makes conventional ion exchange-based processes unfeasible. The present method also permits processing of plant or biomass residual material directly from upstream processing without storage requirements which not only reduces storage and energy requirements, but also significantly reduces susceptibility of the material to microbiological contamination which is common when residual material must be stored for periods of time.

In another aspect of the invention, an apparatus useful to conduct the present protein isolation method is provided. Features of the apparatus are described in U.S. Pat. No. 9,643,175 to Renix Inc., the contents of which are incorporated herein by reference in their entirety. As shown in FIGS. 2A) and 2B), the apparatus comprises a first (adsorber) column 10 and a second (desorber) column 12 interconnected generally by solid transfer and washing systems. First and second fluidized beds are formed in the first and second columns 10, 12, respectively, when protein-capture particles or protein-loaded particles, respectively, are input into each column along with fluids, such as protein-containing feed fluid or eluting fluids. The first adsorber column 10 comprises a first particle-feeding transfer line 17 by which solid protein-capture particles are input into the upper region of the first column 10 (e.g. within the region above the midpoint of the first column 10) via inlet 73 for downward flow within the first column 10. The first column 10 also comprises a fluid-feeding inlet 22 to feed the protein-containing feed fluid into the lower region 24 for upward flow in the first column 10 (e.g. within the region below the midpoint of column 10).

A first transfer line 14 extends from the bottom of the first column 10, with an optional washer, and connects the first column 10 to the upper end of the second column 12 including inlet 74 (protein-loaded particle entry point). Inlet 74 is located at a point above the mid-region of the second column 12. The first transfer line 14 is adapted with a dynamic seal to permit passage of protein-loaded particles and to prevent passage of fluid, e.g. feed fluid, from the first column 10 into the second column 12.

A protein-recovery fluid (eluent) is introduced into the lower end of the second desorber column 12 through one or more second fluid-feeding inlets 26 and is flowed counter-currently to the protein-loaded particles. Protein is released/desorbed from the particles 18 on exposure to the eluent. Protein eluate is removed from the second column 12 through an outlet 63 at the top of the second column 12 located above inlet 74 such that a freeboard section of sufficient height is established between the outlet 63 and inlet 74 to substantially eliminate carryover of particles through the outlet 63.

A second transfer pipe 15, connecting the second column 12 to a transport section 50, extends from the bottom of the second column 12 to the transport section 50, and may also include an optional washer. The second transfer pipe 15 is adapted to be a dynamic seal to permit passage of protein-free particles 18 and prevent passage of protein-containing fluid therethrough.

The transport section carries protein-free particles to a regenerator 46 which functions to regenerate the particles in a third fluidized bed prior to re-entry into the first adsorber column. The regenerator 46 comprises at its upper end, a fluid outlet 65 for removal of separated seffluent-containing fluid, and at its lower end, an outlet 47 for solid regenerated particles for re-entry into the adsorber column. The particles are regenerated by counter-current downward flow of regenerant to replenish the particles to an appropriate chemical state for use to repeat the cycle of protein adsorption, desorption and collection, e.g. by removal of minerals, organic material and the like.

A third transfer pipe 17, connects outlet 47 of the regenerator 46 to the first column 10 and functions to deliver conditioned particles into the top of first fluidized bed within first column 10 via inlet 73. Similar to the other transfer pipes, the third transfer pipe may be adapted to include a dynamic seal in order to prevent transfer of material other than the desired conditioned particles, and may also include a washer to ensure that the particles are suitable for re-entry into the adsorber.

Embodiments of the invention are described in the following specific examples which are not to be construed as limiting.

### Example 1

Protein purification of residual material from the extraction of defatted soy powder was performed according to an embodiment of the present invention. The residual material formed the aqueous protein-containing feed fluid. The feed fluid had a pH of greater of between 8 and 9, and was buffered using 10 mM Na₂PO₄—HCl.

The feed fluid was not pre-treated prior to purification. The feed fluid was fed directly into an apparatus as described herein and illustrated in FIG. 2. Protein in the feed fluid was captured in the adsorber using a weak base anionic resin (such as a resin functionalized with diethylamino groups, e.g. Relisorb DA405, Mitsubishi Chemicals) and recovered in the desorber using a range of concentrations (0.25-1M) of NaCl buffered in 10 mM Na₂PO₄—HCl, pH 10.5 as the protein recovery fluid to yield a protein-rich eluate of high purity. The protein-rich eluate was subjected to ultra-filtration and was then fed into a dryer to produce dried protein.

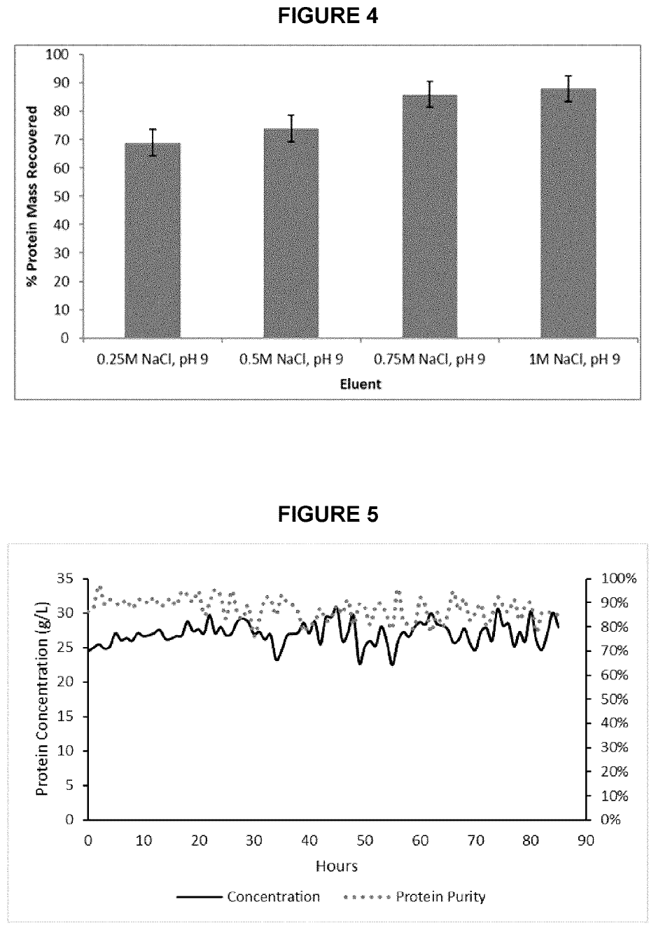

Using this procedure with different concentrations of the protein recovery fluid (0.25M, 0.5M, 0.75M and 1M NaCl), 70-90% of protein mass was recovered from the protein-containing feed fluid as shown in FIG. 4.

The protein-free resin particles were transported to the regenerator of the apparatus for conditioning using HCl at a concentration in the range of 0.01M, and were then fed into the adsorber column of the apparatus to repeat the cycle.

### Example 2

In a second example, residual material from a fermentation process formed the aqueous protein-containing feed fluid. The feed fluid also contained residual carbohydrates, oil, fibre and ash. The feed fluid had a pH between 4 and 5.

The protein-containing feed fluid was pre-treated to separate oil and suspended solids leaving an aqueous solution of carbohydrates, fibre, ash and protein as the feed fluid. The protein-containing feed fluid was fed into the adsorber of an apparatus as in Example 1 comprising RNX-GRAN protein-capture particles (available from Renix Inc.). Proteins were adsorbed onto the particles. In the desorber, a caustic recovery fluid at a concentration of 3.5% was used to elute the protein. The protein-rich eluate was then recovered at between 20 and 30 g/L and a purity of greater than 80% purity as shown in FIG. 5.

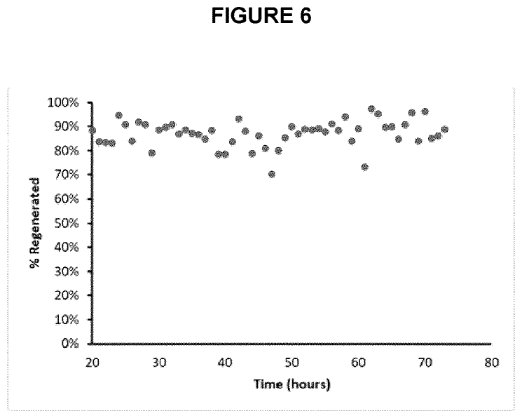

The protein-free particles were circulated into the regenerator and conditioned using an acid at a concentration of 8% to remove minerals from the resin that remain following desorption of the protein. Seffluent comprising the desorbed minerals was removed from the resin. To confirm regeneration of the resin, the mineral content of the seffluent was compared to the theoretical mineral-containing capacity of the resin (concentration of exchange sites X circulation rate) to provide the % regeneration of the resin. Regeneration was determined to be highly effective with an average % regeneration of about 85% (median of about 87%) as shown in FIG. 6. Regenerated resin was flowed back into the adsorber to repeat the cycle.

1. A steady-state process for the recovery of soluble protein from an aqueous protein-containing feed fluid comprising the steps of:
i) passing protein capture particles in a flow countercurrent to the feed fluid in an adsorber at steady state pH to yield protein-loaded particles;
ii) transferring the protein-loaded particles from the adsorber into a desorber;
iii) passing said protein-loaded particles in a flow countercurrent to a protein recovery eluent in the desorber to desorb said protein from said particles at steady state pH to provide protein-free particles;
iv) collecting the desorbed protein from the desorber;
v) transporting the protein-free particles to a regenerator, and passing the protein-free particles countercurrently to a regenerant fluid; and
vi) feeding the regenerated particles into the adsorber for use to repeat steps i)-v) in a continuous manner.

2. The process of claim 1, wherein the protein capture particles are selected from the group of ion exchange particles, adsorbent particles, chromatography particles and zeolite particles.

3. The process of claim 2, wherein the protein capture particles are ion exchange particles.

4. The process of claim 1, wherein the protein recovery eluent is an acid, base or salt.

5. The process of claim 1, wherein the collected desorbed protein is at least 70% pure.

6. The process of claim 5, wherein the collected desorbed protein is at least 80% pure.

7. The process of claim 6, wherein the collected desorbed protein is at least 90% pure.

8. The process of claim 1, wherein the protein-free particles are regenerated by passing the protein-free particles in a flow countercurrent to a regenerant fluid.

9. The process of claim 8, wherein the regenerant fluid is an acid, base or salt.

10. The process of claim 8, wherein the regenerant fluid is selected to release minerals and/or organic material from the protein-free particles.

11. The process of claim 1, wherein the protein-free particles are regenerated at steady state pH.

12. The process of claim 1, wherein the feed fluid has a pH of below 5 or above 7.5.

13. The process of claim 1, wherein the protein capture particles flow downwardly and the feed fluid flows upwardly.

14. The process of claim 1, wherein the protein capture particles are silica-free.

15. The process of claim 1, wherein the protein-loaded particles flow downwardly and the protein-recovery fluid flows upwardly.

16. The process of claim 1, wherein the protein-free particles are flowed downwardly and the regenerant fluid is flowed upwardly.

17. The process of claim 1, wherein the change in pH during protein capture and protein recovery is maintained constant to minimize protein precipitation.

18. An apparatus for use to recover soluble protein at high purity from an aqueous protein-containing feed fluid, said apparatus comprising:
a first column for housing a first fluidized bed, said first column comprising a first particle-feeding inlet to feed protein capture particles into an upper region of said first column and a first fluid-feeding inlet to feed a first protein-containing fluid into a lower region of said first column to form a fluidized bed in the first column, said first column further comprising a first fluid outlet in the upper region at a height above the first particle-feeding means, said lower region being remote from said upper region such that said particles and said feed fluid flow counter-currently, wherein the upper region comprises a first end of the first column and the lower region comprises a second end of the first column;
a second column for housing a second fluidized bed, said second column comprising a second particle-feeding inlet to feed particles into an upper end of said second column and a second fluid-feeding inlet to feed a protein recovery fluid into a lower end of said second column to form a second fluidized bed in the second column, said second column further comprising a second fluid outlet at the upper end of the second column at a height above the second particle-feeding means, said upper and lower ends being remote such that said particles and said fluid flow counter-currently through said second column;
a first conduit which connects the first and second fluidized beds such that the second end of said first column is connected to the upper end of said second column, said first connecting conduit being adapted to permit passage of particles and prevent or minimize passage of said first protein-containing fluid into said second column;
a second conduit which connects the lower end of said second column to a transport section, said second connecting conduit being adapted to permit passage of protein-free particles and prevent or minimize passage of said protein recovery fluid into the transport section; and
a regenerator to which the transport section connects and which is connected to the first particle-feeding inlet of the upper region of the first column via a third conduit, said transport section adapted for hydraulic transport of particles upwardly into the regenerator, which is for housing a third fluidized bed, said regenerator comprising a third particle-feeding inlet to feed protein-free particles into an upper end of said regenerator from the transport section and a third fluid-feeding inlet to feed a regenerant fluid into a lower end of said regenerator to form a third fluidized bed in the regenerator, said regenerator further comprising a third fluid outlet for removal of mineral-containing seffluent at the upper end of the regenerator at a height above the third particle-feeding inlet, said upper and lower ends being remote such that said protein-free particles and said fluid flow counter-currently through said regenerator,
wherein the third conduit connects the lower end of said regenerator to the upper region of said first column, said third connecting conduit being adapted to permit passage of particles and prevent or minimize passage of said regenerant fluid into said first column.
